(19) United States
(12) Patent Application Publication
(10) Pub. No.: US 2025/0205423 A1
(43) Pub. Date: Jun. 26, 2025

(54) DOSE SENSATION EVALUATION METHOD, DOSE SENSATION EVALUATION DEVICE, AND NON-TRANSITORY COMPUTER READABLE STORAGE MEDIUM

(71) Applicants: Akihiro ONISHI, Tokyo (JP); Naomi KURATA, Fujisawa-City (JP); SAWAI PHARMACEUTICAL Co., Ltd., Osaka (JP)

(72) Inventors: Tomohisa KATO, Osaka (JP); Mitsuki YAMAMOTO, Osaka (JP); Akihiro ONISHI, Tokyo (JP); Naomi KURATA, Fujisawa-City (JP)

(21) Appl. No.: 18/652,624

(22) Filed: May 1, 2024

(30) Foreign Application Priority Data
Dec. 20, 2023 (JP) 2023-215059

(51) Int. Cl. A61M 5/172

(52) U.S. Cl. CPC A61M 5/1723; A61M 2230/60

(57) ABSTRACT

A dose sensation evaluation method of a pharmaceutical preparation includes attaching a part of an electromyograph to a human body and acquiring measurement data measured when dosing a pharmaceutical preparation using the attached electromyograph and generating dose sensation evaluation information when dosing a pharmaceutical preparation based on the measurement data. In the dose sensation evaluation method, when measuring the measurement data, the electromyograph may be attached to either the suprahyoid or subhyoid muscle group of the human body.

### CROSS-REFERENCE TO RELATED APPLICATIONS

This application claims the benefit of priority to Japanese Patent Application No. 2023-215059, filed on Dec. 20, 2023, the entire contents of which are incorporated herein by reference.

### FIELD

The present disclosure relates to a new evaluation method of a pharmaceutical preparation. More specifically, the present disclosure relates to a new measurement method, a measurement device, and a non-transitory computer readable storage medium including a program for evaluating a dose sensation based on an amount of muscle activity when dosing a pharmaceutical preparation.

### BACKGROUND

In recent years, a method (papers 5 to 7) of evaluating a swallowing ability and the ease of swallowing objectively using an apparatus and the like is known in addition to the subjective evaluation (papers 1 to 4) by subjects when evaluating the dose sensation of a pharmaceutical preparation.
(Paper 1) Taeyuki Oshima, 7 others, “Effect of Size and Shape of Tablets and Capsules on Ease of Grasping and Swallowing (1): Comparison between Elderly and Students,” Japanese Journal of Pharmaceutical Health Care and Sciences, 2006, Vol. 34, No. 12, p. 842-848
(Paper 2) Yasuo Nakajima, “Comparison of ease of use by capsules vs tablets,” Pharmaceutical Affairs Newspaper, 1991, No. 1636, p. 91-94
(Paper 3) Naomi Kurata, 3 others, “Medicines That are Appropriate for Elderly Persons-Evaluation of The Size of Oral Disintegrating Tablets That can be Ingested-” Japanese Journal of Pharmaceutical Health Care and Sciences, 2010, Vol. 36, No. 6, p. 397-405
(Paper 4) Hiroko Miura, 1 other, “Effect of size of tablets on easiness of swallowing and handling among the frail elderly,” Japanese Journal of Geriatrics, 2007, Vol. 44, p. 627-633
(Paper 5) Toshio Matsuoka, 3 others, “Evaluation of Intake Easiness for Tablet,” The Japanese Journal of Ergonomics, 2007, Vol. 43, p. 444-445
(Paper 6) Takahiro Ono, 5 others, “What tongue pressure means in mastication and swallowing,” The Japanese Journal of Dysphagia Rehabilitation, 2006, Vol. 10, No. 3, p. 207-219
(Paper 7) Takahiro Ono, 5 others, “Quantitative Evaluation of Swallowing Function of Patients with Parkinson's Disease Using a Tongue Pressure Sensor Sheet”, Journal of the Society of Biomechanics, 2010, Vol. 34, No. 2, p. 105-110

### SUMMARY

According to the present disclosure, a dose sensation evaluation method of a pharmaceutical preparation includes attaching a part of an electromyograph to a human body and acquiring measurement data measured when dosing a pharmaceutical preparation using the attached electromyograph, and generating dose sensation evaluation information when dosing a pharmaceutical preparation based on the measurement data.

In the dose sensation evaluation method, the electromyograph may be attached to either the suprahyoid or subhyoid muscle group of the human body when dosing the pharmaceutical preparation.

The dose sensation evaluation method may further include, after acquiring the measurement data, performing a root-mean-square process of first data contained in the measurement data to generate second data, generating third data by adopting points from the second data, the points corresponding to the second data and exceeding an average value of a baseline plus twice a standard deviation when not dosing the pharmaceutical preparation, and generating the dose sensation evaluation when information dosing the pharmaceutical product based on the third data.

In the dose sensation evaluation method, the dose sensation evaluation information when dosing the pharmaceutical preparation may be generated based on the maximum value of the potential in the third data.

In the dose sensation evaluation method, the dose sensation evaluation information when dosing the pharmaceutical preparation may be generated based on an area value generated from the third data.

The dose sensation evaluation method may further include determining a good dose sensation when the dose sensation evaluation information meets a predetermined condition.

The dose sensation evaluation method may further include notifying a determination result of the dose sensation.

In the dose sensation evaluation method, the pharmaceutical preparation may be one of capsules, tablets, granules, and powders.

According to the present disclosure, a non-transitory computer readable storage medium storing a program for causing a computer to execute the above dose sensation evaluation method of a pharmaceutical preparation is provided.

According to the present disclosure, a dose sensation evaluation device includes a processor, and a memory configured to store a program, the program executed by the processor to cause the processor to attach a part of an electromyograph to a human body and acquire measurement data measured when dosing a pharmaceutical preparation using the attached electromyograph, and generate dose sensation evaluation information when dosing the pharmaceutical preparation based on the measurement data.

In the dose sensation evaluation method, the electromyograph may be attached to either the suprahyoid or subhyoid muscle group of the human body when dosing the pharmaceutical preparation.

In the dose sensation evaluation method, the program may cause the processor to after acquiring the measurement data, perform a root-mean-square process of first data contained in the measurement data to generate second data, generate third data by adopting points from the second data, the points corresponding to the second data and exceeding an average value of a baseline plus twice a standard deviation when not dosing the pharmaceutical preparation, and generate the dose sensation evaluation information when dosing the pharmaceutical product based on the third data.

In the dose sensation evaluation method, the dose sensation evaluation information when dosing the pharmaceutical preparation may be generated based on the maximum value of the potential in the third data.

In the dose sensation evaluation method, the dose sensation evaluation information when dosing the pharmaceutical preparation may be generated based on an area value generated from the third data.

In the dose sensation evaluation method, the program may cause the processor to determine a good dose sensation when the dose sensation evaluation information meets a predetermined condition.

In the dose sensation evaluation method, the program may cause the processor to notify a determination result of the dose sensation.

In the dose sensation evaluation method, the pharmaceutical preparation may be one of capsules, tablets, granules, and powders.

According to the present disclosure, it is possible to provide a dose sensation evaluation method of a pharmaceutical preparation that can be quantitatively evaluated.

### BRIEF DESCRIPTION OF DRAWINGS

FIG. 1 is a diagram showing a configuration of hardware in a dose sensation evaluation system according to an embodiment of the present disclosure.

FIG. 2 is a diagram for explaining a software configuration of a dose sensation evaluation system according to an embodiment of the present disclosure.

FIG. 3 is a flowchart showing a dose sensation evaluation method of a dose sensation evaluation system according to an embodiment of the present disclosure.

FIG. 4 is a flowchart showing a dose sensation evaluation method of a dose sensation evaluation system according to an embodiment of the present disclosure.

FIG. 5 is a flowchart showing a dose sensation evaluation method of a dose sensation evaluation system according to an embodiment of the present disclosure.

FIG. 6 is an example of measurement data according to an embodiment of the present disclosure.

FIG. 7 is an example of measurement data according to an embodiment of the present disclosure.

FIG. 8 is an example of measurement data according to an embodiment of the present disclosure.

FIG. 9 is an example of measurement data according to an embodiment of the present disclosure.

FIG. 10 is a graph showing a dose sensation evaluation of a pharmaceutical preparation based on an impression before the dose of the pharmaceutical preparation (questionnaire).

FIG. 11 is a graph showing a dose sensation evaluation of a pharmaceutical preparation based on an impression after the dose of the pharmaceutical preparation (questionnaire).

FIG. 12 shows a result of a correlation evaluation between a dose sensation before the dose of the pharmaceutical preparation and a dose sensation after the dose of the pharmaceutical preparation.

FIG. 13 is a photograph showing the position to which the electromyograph is attached.

FIG. 14 shows a dose sensation evaluation result of a pharmaceutical preparation based on the electromyography measurement in the suprahyoid muscle group.

FIG. 15 shows a dose sensation evaluation result of a pharmaceutical preparation based on the electromyography measurement in the subhyoid muscle group.

FIG. 16 is a correlation evaluation result of the questionnaire after the dose and the dose sensation evaluation of a pharmaceutical preparation by the electromyography measurement.

### DESCRIPTION OF EMBODIMENTS

The following embodiments are examples of embodiments of the present disclosure, and the present disclosure is not limited to these embodiments. In the drawings referred to in the present embodiment, the same or similar parts are denoted by the same reference signs or similar reference signs, and repeated description thereof may be omitted.

It is difficult to maintain objectivity and reproducibility in the subjective evaluation by the subject which is widely used at present. Therefore, an objective method using a device or the like is desired, but an established method is not yet known.

In view of the above, an object of the present disclosure is to provide a dose sensation evaluation method of a pharmaceutical preparation that can be quantitatively evaluated.

A dose sensation evaluation system according to an embodiment of the present disclosure will be described in detail with reference to the drawings.

### [1-1. Hardware Configuration of Dose Sensation Evaluation System]

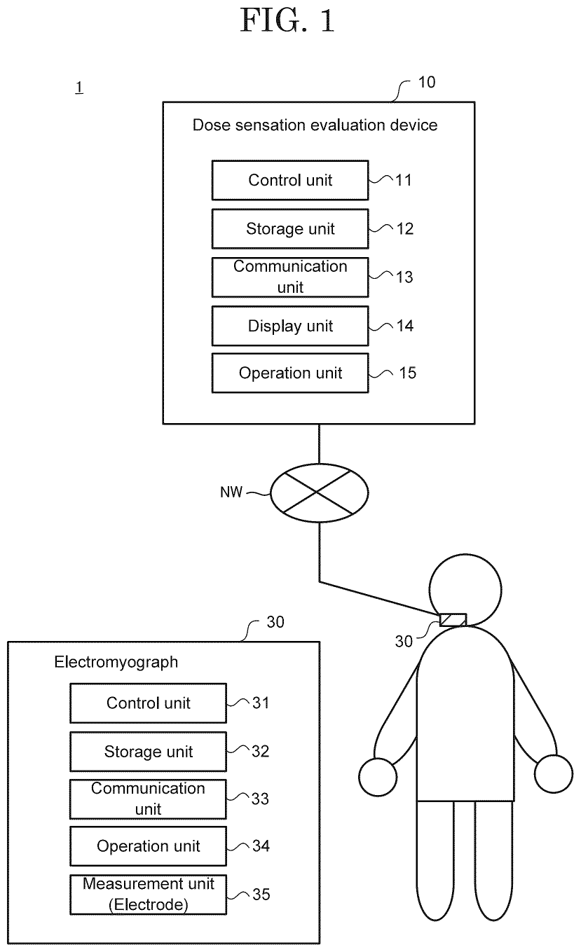

FIG. 1 shows a hardware configuration of a dose sensation evaluation system 1. As shown in FIG. 1, the dose sensation evaluation system 1 includes a dose sensation evaluation device 10 and an electromyograph 30. The dose sensation evaluation device 10 can also function as a database and an application server for dose sensation evaluation. The electromyograph 30 is a device that is attached to a human body and measures a change in potential that occurs when dosing a pharmaceutical preparation. Although the case where one electromyograph 30 is used is shown for convenience of explanation in the present embodiment, a plurality of electromyographs 30 may be used as appropriate.

According to the dose sensation evaluation system 1, the electromyograph 30 transmits measurement data (data indicating the variation in the potential) measured when dosing a pharmaceutical preparation to the dose sensation evaluation device 10 via a network NW. The dose sensation evaluation device 10 analyzes the measurement data. The dose sensation evaluation device 10 then generates dose sensation evaluation information from the analysis result and determines the dose sensation based on the dose sensation evaluation information (result). Hereinafter, a configuration of a dose sensation evaluation system for realizing such a process will be described.

The dose sensation evaluation device 10 includes a control unit 11, a storage unit 12, a communication unit 13, a display unit 14, and an operation unit 15. The dose sensation evaluation device 10 may be used as a server. In this case, the dose sensation evaluation device 10 may be either an on-premises server or a cloud server.

The control unit 11 is a type of computer, and controls a process based on a command defined in the dose sensation evaluation control software (program) which will be described later, using a CPU (Central Processing Unit), an ASIC (Application Specific Integrated Circuit), an FPGA (Field Programable Gate Array), or other calculation processing circuits. In addition, a user interface for executing a dose sensation evaluation control program may be provided to the display unit 14 by commands from the control unit 11.

A semiconductor memory such as an SSD (Solid State Drive), magnetic recording media (magnetic tapes, magnetic disks, and the like), optical recording media, magneto-optical recording media, and memorizable elements that are storage media are used in the storage unit 12. The storage unit 12 has a function as a database for storing the dose sensation evaluation control information used in a dose sensation evaluation control program. The storage unit 12 may be arranged in a device different from the dose sensation evaluation device 10 as appropriate, and may function as a database.

The communication unit 13 has a transceiver and transmits and receives measurement data to and from the electromyograph 30 via the network NW. Communication between the communication unit 13 and the electromyograph 30 is performed by wire or wirelessly.

The operation unit 15 includes a controller, a button, or a switch. When an operation such as moving up, down, left, or right, pressing, or rotating is performed by the operation unit 15, data based on such operations is transmitted to the control unit 11. Further, in the case of a display device (touch panel) having a touch sensor, the display unit 14 and the operation unit 15 may be arranged at the same position.

Although not shown in the drawings, the dose sensation evaluation device 10 may include a sound output unit (buzzer) and an illumination unit (light).

The electromyograph 30 includes a control unit 31, a storage unit 32, a communication unit 33, an operation unit 34, and a measurement unit 35. The electromyograph 30 is used by being attached to either the suprahyoid muscle group or the subhyoid muscle group of the human body.

The control unit 31 is a type of computer and includes a CPU, an ASIC, an FPGA, or other calculation processing circuits. The control unit 31 causes dose sensation evaluation related programs stored in the storage unit 32 such as a memory to be executed based on the operation of the operation unit 34, and instructs the execution of the processing related to a dose sensation evaluation control processing program stored in the storage unit 12 of the dose sensation evaluation device 10.

The communication unit 33 has a function of transmitting and receiving data to and from the dose sensation evaluation device 10. For example, a LAN transceiver (for example, a Wi-Fi transceiver) is used for the communication unit 33. In addition, the transceiver is not limited to the LAN transceiver, and a transceiver for portable terminal communication (for example, LTE communication) may be used, or a transceiver for short-range radio communication may be used. The electromyograph 30 is connected to the dose sensation evaluation device 10 via the network NW.

The operation unit 34 includes a controller, a button, or a switch. When an operation such as moving up, down, left, or right, pressing, or rotating is performed by the operation unit 34, data based on such operations is transmitted to the control unit 31.

The measurement unit 35 includes an electrode to be attached to the human body. The electrode has any form (shape), such as a needle electrode, a wire electrode, or a surface electrode.

### [1-2. Software Configuration of Dose Sensation Evaluation System]

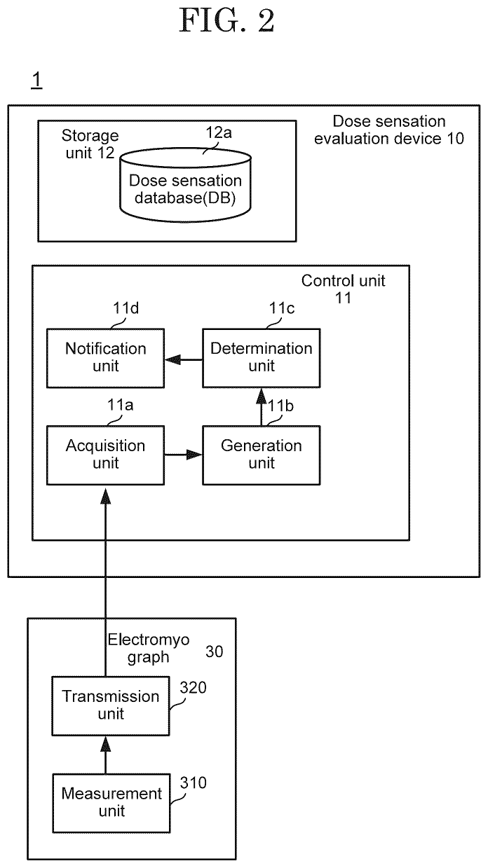

FIG. 2 is a diagram showing a software configuration of the dose sensation evaluation system 1. The control unit 11 of the dose sensation evaluation device 10 includes an acquisition unit 11a, a generation unit 11b, a determination unit 11c, and a notification unit 11d.

The acquisition unit 11a has a function of acquiring measurement data from the electromyograph 30. In addition, the acquisition unit 11a may acquire the measurement data stored in advance in a dose sensation database (DB) 12a of the storage unit 12.

The generation unit 11b has a function of generating various types of information based on the measurement data acquired by the acquisition unit 11a. In this example, the generation unit 11b generates the dose sensation evaluation information.

The determination unit 11c has a function of determining whether the acquired information satisfies a predetermined condition. In this example, the determination unit 11c determines whether the generated dose sensation evaluation information exceeds a threshold value.

The notification unit 11d has a function of notifying the determination result of the dose sensation to the exterior of the unit. In this example, the determination result may be displayed on the display unit 14 of the dose sensation evaluation device 10, may be transmitted to an external device, or may be output as sound or light.

The electromyograph 30 includes a measurement unit 310 and a transmission unit 320. The measurement unit 310 has a function of measuring a variation in the potential at the attached portion. The transmission unit 320 has a function of transmitting the measured potential (measurement data) to the dose sensation evaluation device 10.

### [1-3. Dose Sensation Evaluation Method]

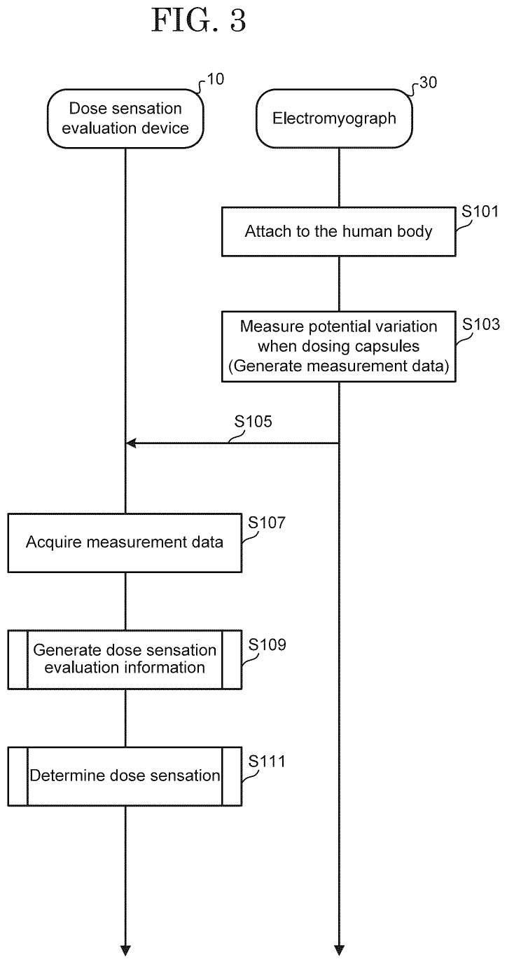

Next, a dose sensation evaluation method realized in the dose sensation evaluation system according to an embodiment of the present disclosure will be described. FIG. 3 to FIG. 5 are flowcharts showing a dose sensation evaluation method of the dose sensation evaluation system 1 according to the present embodiment.

First, the electromyograph 30 is attached to the human body (step S101). The electromyograph 30 is used by being attached to either the suprahyoid muscle group or the subhyoid muscle group of the human body.

When evaluating the dose sensation using the electromyograph 30, recording is started according to a signal. Next, a person (subject) doses the pharmaceutical preparation based on a dose signal 5 seconds after the start of recording. The measurement is continued for 5 seconds after the dose. This series of data is used as measurement data (step S103). The frequency band used in the measurement is 50 to 450 Hz. The measurement data measured by the electromyograph 30 is transmitted to the dose sensation evaluation device 10 by wire or wirelessly (step S105).

Next, the control unit 11 of the dose sensation evaluation device 10 acquires the transmitted measurement data (step S107). The acquired measurement data is stored in the dose sensation evaluation DB 12a.

Next, the dose sensation evaluation device 10 generates dose sensation evaluation information based on the measurement data (step S109). Hereinafter, a method of generating the dose sensation evaluation information will be described in detail.

### [Generation of Dose Sensation Evaluation Information]

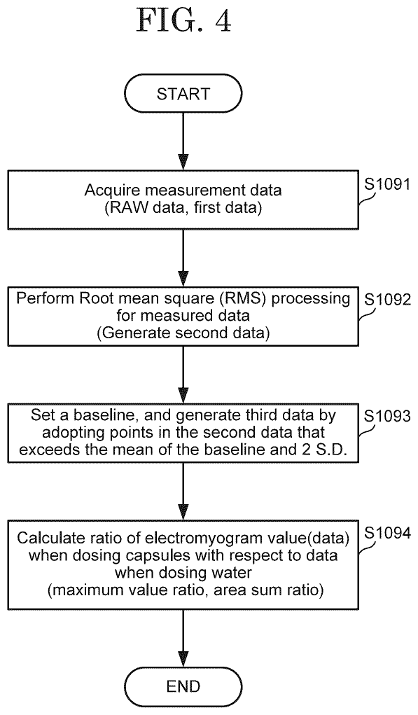

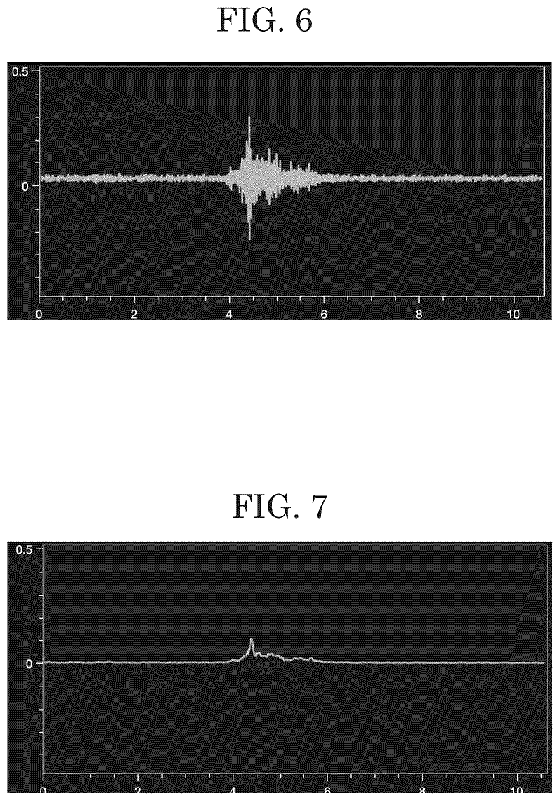

FIG. 4 is a flowchart of the generation of the dose sensation evaluation information. First, the dose sensation evaluation device 10 acquires the measurement data (step S1091). In this case, the measurement data includes RAW data (first data) composed of potential values at respective times as shown in FIG. 6. The RAW data includes a positive potential value and a negative potential value.

Next, the dose sensation evaluation device 10 performs a root mean square (RMS) process on the RAW data (generates second data) (step S1092). As a result, the second data indicates a positive potential value, as shown in FIG. 7.

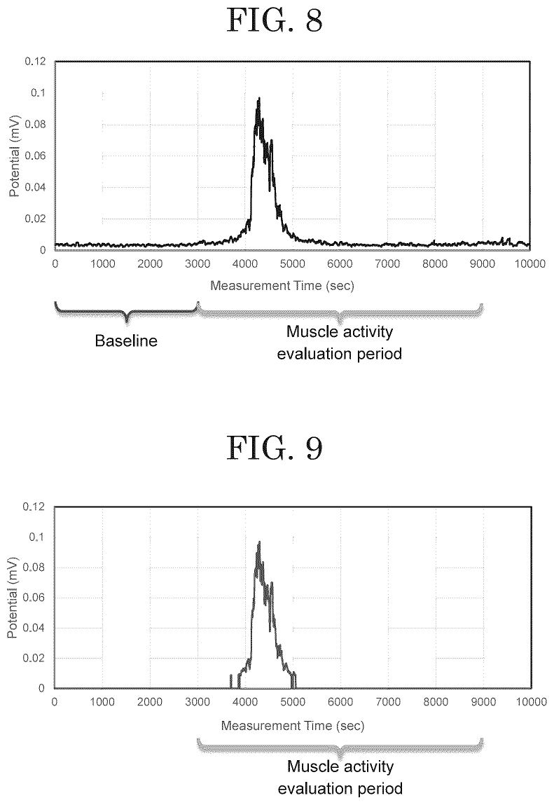

Next, as shown in FIG. 8, the dose sensation evaluation device 10 sets a baseline (in this case, 0 to 3000 sec) with respect to the root-mean-square processed data (second data), sets a point (having a higher potential value) exceeding a value (average value plus 2S.D.) acquired by adding a double value of the standard deviation (S.D.) to the average value of the baseline (also referred to as “base value”) from the second data as a muscle activity evaluation point (corresponding to the muscle activity evaluation period of 3000 to 9000 sec), and sets the value as third data (step S1093). As a result, the numerical value other than when dosing the pharmaceutical preparation is “0”, as shown in FIG. 9.

The third data is then used to generate the maximum value and an area sum value. A value (ratio) is calculated by dividing the maximum value and the area sum value by the data (maximum value, area sum value) when drinking water (step S1094). These are used as the dose sensation evaluation information.

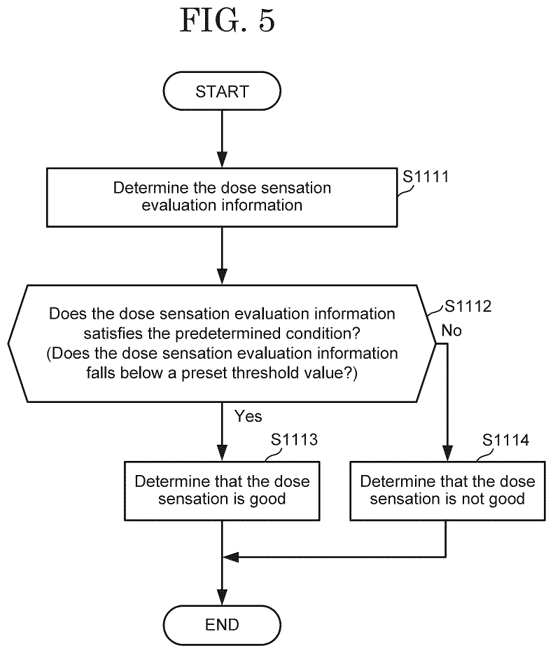

Returning to FIG. 3, next, the dose sensation evaluation device 10 determines the dose sensation by using the dose sensation evaluation information (step S111). FIG. 5 is a flowchart of a determination process. First, the dose sensation evaluation device 10 determines the dose sensation based on whether the dose sensation evaluation information satisfies a predetermined condition (step S1111). The predetermined condition is that the maximum value ratio (or the area sum ratio) among the dose sensation evaluation information falls below a preset threshold value. In the case where the maximal value ratio (or the area sum ratio) of the dose sensation evaluation information satisfies the predetermined condition (step S1112; Yes), it is determined that the dose sensation is good (step S1113). In the case where the maximal value ratio (or the area sum ratio) of the dose sensation evaluation information does not satisfy the predetermined condition (step S1112; No), it is determined that the dose sensation is not good (step S1114).

The determination result of the dose sensation may be notified to the exterior of the unit. In this example, the determination result may be displayed on the display unit 14 of the dose sensation evaluation device 10, may be transmitted to the external device, or may be output as sound or light. The above description is the dose sensation evaluation method. In the case of the above-described dose sensation evaluation method, quantitative data such as a potential value is used. Therefore, the dose sensation of the pharmaceutical preparation can be quantitatively evaluated rather than organoleptically.

### Modifications

The present disclosure is not limited to the above-described embodiments, and includes various other modifications. For example, the above-described embodiments have been described in detail for the purpose of illustrating the present disclosure in an easy-to-understand manner, and are not necessarily limited to those having all the described configurations. In addition, part of the configuration of an embodiment may be replaced with the configuration of another embodiment, and the configuration of another embodiment may be added to the configuration of an embodiment. In addition, it is possible to add, delete, or replace part of the configuration of each embodiment with another configuration.

Although the example in which the dose sensation evaluation device 10 and the electromyograph 30 are separately arranged has been described in an embodiment of the present disclosure, the present disclosure is not limited to this. The dose sensation evaluation device 10 and the electromyograph 30 may be integrally arranged.

Although an example in which a capsule is used as the pharmaceutical preparation is shown in an embodiment of the present disclosure, the present disclosure is not limited to this. For example, any tablets, granules, and powders in addition to the capsule can be used as the pharmaceutical preparation to achieve a similar effect.

### Examples

Next, the present disclosure will be described in more detail based on the examples. In addition, the following description is intended to facilitate understanding of the present disclosure, but the present disclosure is not limited to this. That is, modifications, embodiments, and other examples based on the technical idea of the present disclosure are all included in the present disclosure.

### [1. Dose Sensation Evaluation of Pharmaceutical Preparation Based on Impressions Before Dose (Questionnaire)]

First, the dose sensation evaluation was carried out on the pharmaceutical preparation based on the impression before the dose. This evaluation was carried out in 10 grades by a questionnaire regarding the “dose sensation” which is the easiness of dosing the capsules judged by looking at and touching the capsules before the dose. Evaluation conditions are as follows.
Subjects: Japanese healthy adults
Age: 20 to 39 years old
BMI (male): 19 to 24.9
BMI (female): 18.5 to 24.9
Male: Female (1:1)
Number of subjects: 40
Preparation used: Placebo preparation (lactose) No. 0 to No. 5 capsules HPMC

Test content: The impression “easy to dose” which was judged by looking and touching is evaluated in 10 grades (specifically, the case in which it seems to be very difficult to dose and there is resistance is evaluated as 0, and the case in which it is easy to dose and there is no resistance at all is evaluated as 10).

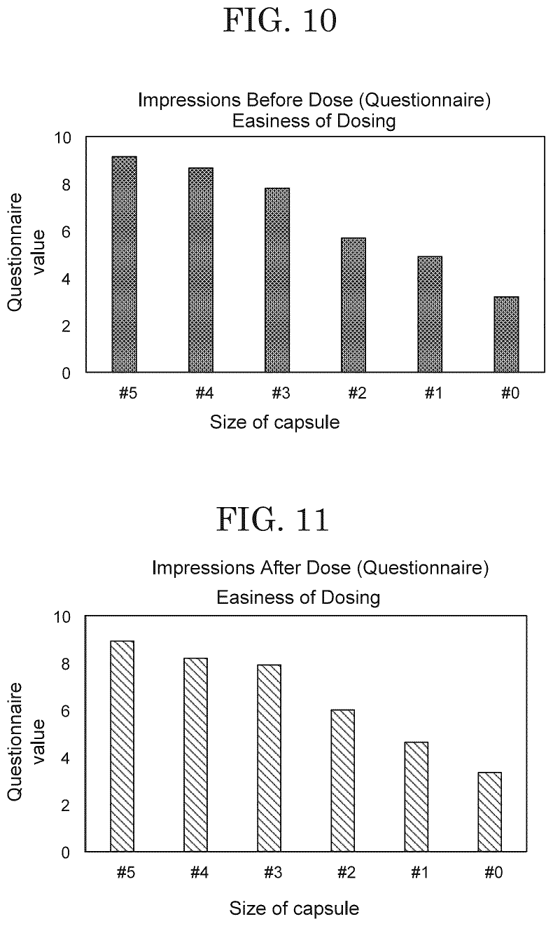

FIG. 10 is a graph showing the dose sensation evaluation of a pharmaceutical preparation based on the impression before the dose (questionnaire). As shown in FIG. 10, in the dose sensation evaluation of the pharmaceutical preparation by impression, the impression of ease of dosing tended to be better (the numerical value became high) as the size of the capsule became smaller.

[2. Dose Sensation Evaluation of Pharmaceutical Preparation Based on Impression after Dose (Questionnaire)]

Next, the dose sensation evaluation was carried out on the pharmaceutical preparation based on the impression after the dose (questionnaire). This evaluation was carried out in 10 grades by a questionnaire regarding the ease of dosing the capsules (i.e., dose sensation) as actually felt by the subject when dosing the capsules. Evaluation conditions are as follows.
Subjects: Japanese healthy adults
Age: 20 to 39 years old
BMI (male): 19 to 24.9
BMI (female): 18.5 to 24.9
Male: Female (1:1)
Number of subjects: 40
Preparation used: Placebo preparation (lactose) No. 0 to No. 5 capsules HPMC
Dosage Method: Dosing 1 capsule with 20 ml water (6 times in total)
   Dosage interval: Approximately 5 minutes interval
   Posture when dosing: Seated on a chair, the orientation of the jaw is horizontal
Test content: The impression “easy to dose” when actually dosing is evaluated in 10 grades (specifically, the case in which it was very difficult to dose and there was resistance is evaluated as 0, and the case in which it was easy to dose and there was no resistance is evaluated as 10).

FIG. 11 is a graph showing the dose sensation evaluation of the pharmaceutical preparation based on the impression after the dose (questionnaire). As shown in FIG. 11, in the dose sensation evaluation of the pharmaceutical preparation based on the impression after the dose, there was a tendency that the dose sensation was better (the numerical value became higher) as the capsule became smaller.

### [3. Correlation of Dose Sensation Evaluation of Pharmaceutical Preparation Based on Impression Before and After the Dose]

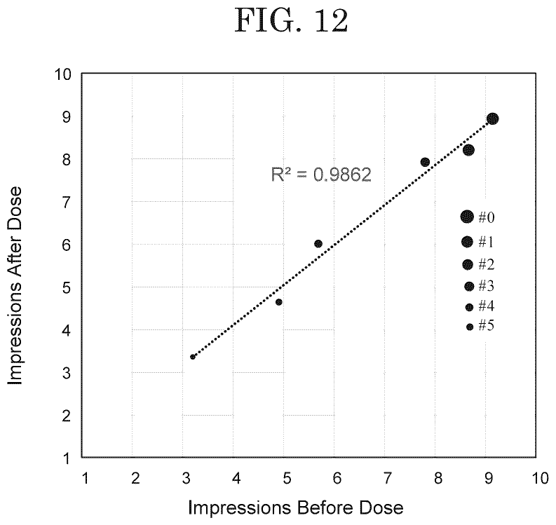

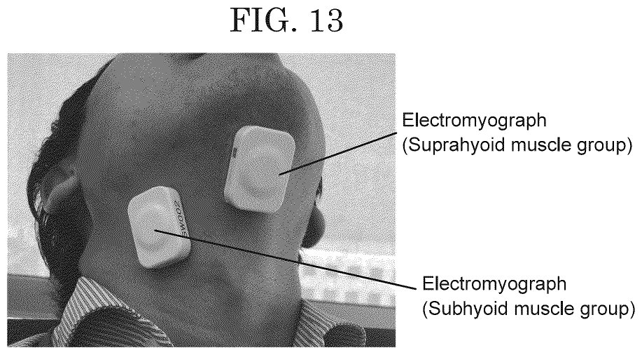

A correlation evaluation was performed on the dose sensation evaluation result of the pharmaceutical preparation based on the impression before and after the dose. FIG. 12 is a correlation evaluation result between the impression before the dose and the impression after the dose. As shown in FIG. 12, there was a high correlation between the dose sensation evaluation result based on the impression before the dose and the dose sensation evaluation result based on the impression after the dose. In other words, it was confirmed that there was a high correlation between the judgment by visual (tactile) information and the actual dose sensation.

### [4. Dose Sensation Evaluation of Pharmaceutical Preparation Based on Electromyography Measurement)

Next, the dose sensation evaluation was carried out of the pharmaceutical preparation based on the electromyography measurement. This evaluation was performed by attaching the electromyograph (manufactured by Trunk Solution Corporation) to subjects. The evaluation conditions are as follows.
Subjects: Japanese healthy adults
Age: 20 to 39 years old
BMI (male): 19 to 24.9
BMI (female): 18.5 to 24.9
Male: Female (1:1)
Number of subjects: 40
Formulations used: Placebo preparation (lactose) No. 0 to No. 5 capsules HPMC
Dosage Method: Dosing 1 capsule with 20 ml water (6 times in total)
   Dosage interval: Approximately 5 minutes interval
   Posture when dosing: Seated on a chair, the orientation of the jaw is horizontal
Test content: The muscle activity of the muscles when swallowing is measured using the electromyograph. The electromyography was attached to the suprahyoid muscle group and the subhyoid muscle group (see FIG. 13).
Analysis method: The result of dosing only 20 ml of water was set to 1.00, and the ratio (maximum value ratio, area sum ratio) of an electromyogram value when dosing capsules with respect to those when dosing water was calculated. The condition of dosing 20 ml of water was measured three times, and the average of three times was used. T-test (bilateral, corresponding) was performed between the preparations, and a significance test was performed at a significance level of 0.05.

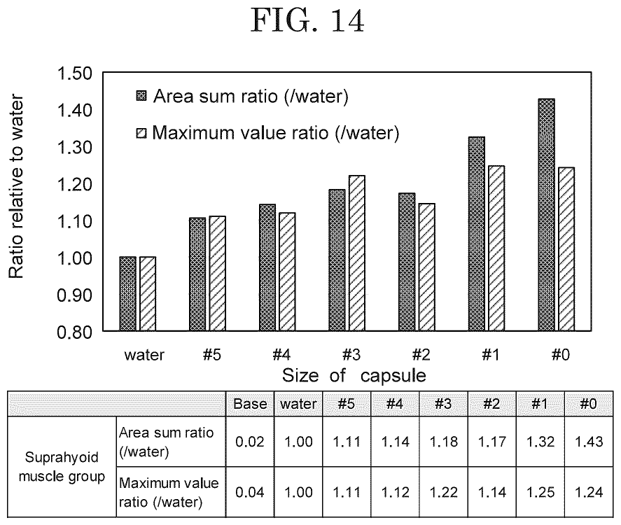

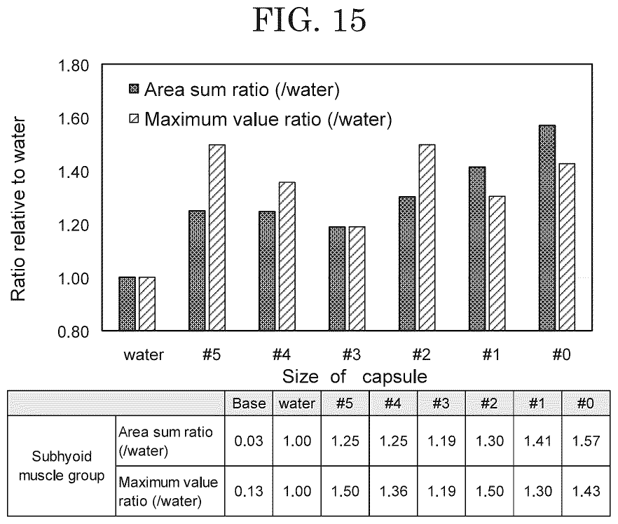

FIG. 14 is a result of the dose sensation evaluation of a pharmaceutical preparation based on the electromyography measurement in the suprahyoid muscle group. FIG. 15 is a result of the dose sensation evaluation of a pharmaceutical preparation based on the electromyography measurement in the subhyoid muscle group. As shown in FIG. 14, an increase in the numerical value correlated with the size of the capsule was observed in the dose sensation evaluation result in which the electromyograph is attached to the suprahyoid muscle group. In addition, as shown in FIG. 15, although there was a variation in changes of the value when the size of the capsule was small in the results of the dose sensation evaluation in which the electromyograph is attached to the subhyoid muscle group, an increase in the numerical value roughly corresponding to the size of the capsule was observed.

[5. Correlation Between Impression after Dose and Dose Sensation Evaluation of Pharmaceutical Preparation by Electromyography Measurement]

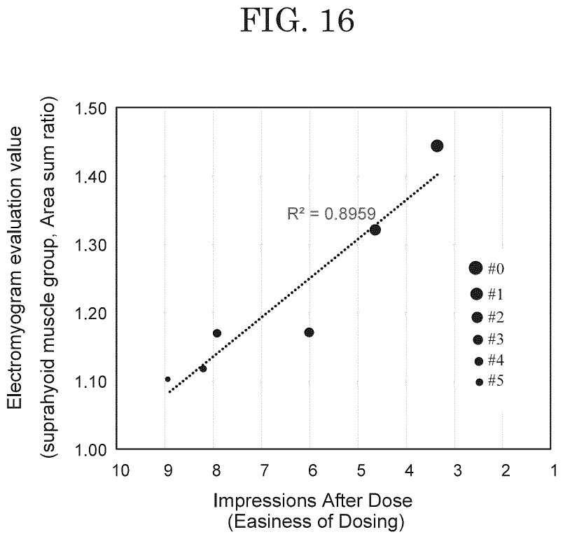

Correlation evaluation was carried out for the impression after the dose (questionnaire) and the dose sensation evaluation result of the pharmaceutical preparation by the electromyography measurement. In addition, the subjects in the evaluation by the questionnaire are the same as the subjects in the evaluation electromyography measurement. FIG. 16 is a correlation evaluation result between the impression after the dose (questionnaire) and the dose sensation evaluation of a pharmaceutical preparation by the electromyography measurement. The area sum ratio when the electromyograph was attached to the suprahyoid muscle group was used as the electromyography measurement result. As shown in FIG. 16, there was a high correlation between the results of the questionnaire on the dose sensation and the electromyogram evaluation value (suprahyoid muscle group, area sum ratio). That is, it was confirmed that the electromyography measurement correlates with the actual difficulty in dosing (dose sensation) and is useful as a quantitative evaluation method in the dose sensation of the pharmaceutical preparation.

What is claimed is:

1. A dose sensation evaluation method of a pharmaceutical preparation comprising:
attaching a part of an electromyograph to a human body and acquiring measurement data measured when dosing a pharmaceutical preparation using the attached electromyograph; and
generating dose sensation evaluation information when dosing a pharmaceutical preparation based on the measurement data.

2. The dose sensation evaluation method of a pharmaceutical preparation according to claim 1, wherein
the electromyograph is attached to either the suprahyoid or subhyoid muscle group of the human body when measuring the measurement data.

3. The dose sensation evaluation method of a pharmaceutical preparation according to claim 1 further comprising:
after acquiring the measurement data,
performing a root-mean-square process of first data contained in the measurement data to generate second data;
generating third data by adopting points from the second data, the points corresponding to the second data and exceeding an average value of a baseline plus twice a standard deviation when not dosing the pharmaceutical preparation; and
generating the dose sensation evaluation information when dosing the pharmaceutical preparation based on the third data.

4. The dose sensation evaluation method of a pharmaceutical preparation according to claim 3, wherein
the dose sensation evaluation information when dosing the pharmaceutical preparation is generated based on the maximum value of a potential in the third data.

5. The dose sensation evaluation method of a pharmaceutical preparation according to claim 3, wherein
the dose sensation evaluation information when dosing the pharmaceutical preparation is generated based on an area value generated from the third data.

6. The dose sensation evaluation method of a pharmaceutical preparation according to claim 1 further comprising:
determining good dose sensation when the dose sensation evaluation information satisfies a predetermined condition.

7. The dose sensation evaluation method of a pharmaceutical preparation according to claim 6 further comprising:
notifying a determination result of the dose sensation.

8. The dose sensation evaluation method of a pharmaceutical preparation according to claim 1, wherein
the pharmaceutical preparation is one of capsules, tablets, granules, and powders.

9. A non-transitory computer readable storage medium storing a program for causing a computer to execute the dose sensation evaluation method of a pharmaceutical preparation according to claim 1.

10. A dose sensation evaluation device comprising:
a processor; and
a memory configured to store a program, the program executed by the processor to cause the processor to:
attach a part of an electromyograph to a human body and acquire measurement data measured when dosing a pharmaceutical preparation using the attached electromyograph; and
generate dose sensation evaluation information when dosing the pharmaceutical preparation based on the measurement data.

11. The dose sensation evaluation device of a pharmaceutical preparation according to claim 10, wherein
the electromyograph is attached to either the suprahyoid or subhyoid muscle group of the human body when measuring the measurement data.

12. The dose sensation evaluation device of a pharmaceutical preparation according to claim 10, wherein the program causes the processor to
after acquiring the measurement data,
perform a root-mean-square process of first data contained in the measurement data to generate second data;
generating third data by adopting points from the second data, the points corresponding to the second data and exceeding an average value of a baseline plus twice a standard deviation when not dosing the pharmaceutical preparation; and
generate the dose sensation evaluation information when dosing the pharmaceutical preparation based on the third data.

13. The dose sensation evaluation device of a pharmaceutical preparation according to claim 12, wherein
the dose sensation evaluation information when dosing the pharmaceutical preparation is generated based on the maximum value of a potential in the third data.

14. The dose sensation evaluation device of a pharmaceutical preparation according to claim 12, wherein
the dose sensation evaluation information when dosing the pharmaceutical preparation is generated based on an area value generated from the third data.

15. The dose sensation evaluation device of a pharmaceutical preparation according to claim 10, wherein the program causes the processor to
determine a good dose sensation when the dose sensation evaluation information meets a predetermined condition.

16. The dose sensation evaluation device of a pharmaceutical preparation according to claim 15, wherein the program causes the processor to
notify a determination result of the dose sensation.

17. The dose sensation evaluation device of a pharmaceutical preparation according to claim 10, wherein
the pharmaceutical preparation is one of capsules, tablets, granules, and powders.
